(19) United States
(12) Patent Application Publication
(10) Pub. No.: US 2019/0388513 A1
(43) Pub. Date: Dec. 26, 2019

(54) OIL BASED FORMULATIONS FOR SUBLINGUAL AND BUCCAL DELIVERY

(71) Applicant: BIOLINGUS IP LLC, Hergiswil NW (CH)

(72) Inventor: SaiYing KO, Burwood, Victoria (AU)

(21) Appl. No.: 16/487,669

(22) PCT Filed: Feb. 28, 2018
(86) PCT No.: PCT/AU2018/050177

§ 371 Date: Aug. 21, 2019

(30) Foreign Application Priority Data
Feb. 28, 2017 (AU) 2017900677

(51) Int. Cl. A61K 38/28; A61K 9/00; A61K 47/44; A61K 47/22; A61K 47/34; A61K 38/22; A61K 38/26; A61P 3/10; A61K 31/05

(52) U.S. Cl. CPC A61K 38/28; A61K 9/006; A61K 47/44; A61K 47/22; A61K 31/05; A61K 38/2278; A61K 38/26; A61P 3/10; A61K 47/34

(57) ABSTRACT

The present invention is a delivery system for sublingual and/or buccal delivery comprising at least one functional oil (i.e. acting as an oil delivery base); at least one surfactant; and at least one pharmaceutically active agent. The invention also includes a method for treating diabetes, for regulating blood glucose levels and/or for treating hyperglycaemia by sublingual or buccal administration of the delivery system where the pharmaceutical agent is insulin and/or an insulin analogue or mimetic, and/or a glucagon-like peptide-1 agonist.

### FIELD OF THE INVENTION

The present disclosure relates generally to oil-based delivery systems and compositions formulated for the delivery of pharmaceutically active agents via the sublingual and buccal mucosa, and to uses thereof.

### BACKGROUND OF THE INVENTION

Oral formulations for the delivery of active pharmaceutical ingredients, for example proteins and peptides, include tablets, capsules (hard and soft shelled), lozenges, powders, emulsions and liquids. In order to benefit from such formulations the active pharmaceutical ingredient must remain bioavailable after passing through acid digestion in the stomach and enzymatic digestion in the gastrointestinal tract. Daily food consumption also influences the bioavailability of active pharmaceutical ingredients, and the subject must have a well-functioning gastrointestinal system to ensure adequate absorption via the gastrointestinal tract.

To avoid these issues with oral administration, many active pharmaceutical ingredients, for example insulin, are typically administered subcutaneously (e.g. via needle and syringe, and the like, such as pens, jet injectors and pumps). However, many people find such administration devices to be daunting, uncomfortable, painful and/or generally inconvenient. Due to these issues, patient compliance can be an ongoing challenge.

It would therefore be beneficial to produce formulations that can be administered by simpler and less invasive means, while maintaining the bioavailability of the active ingredient.

### SUMMARY OF THE INVENTION

The present inventors have found that transmucosal delivery systems comprising at least one functional oil; at least one surfactant; and at least one active agent can be successfully administered via the sublingual and buccal mucosa, thereby bypassing the acidic environment of the stomach and enzymatic digestion in the gastrointestinal tract, and avoiding the need to break the skin.

A first aspect of the present disclosure provides a transmucosal delivery system formulated for the administration of at least one pharmaceutically active agent via the sublingual and/or buccal mucosa comprising: at least one functional oil; at least one surfactant; and at least one pharmaceutically active agent. In particular embodiments the pharmaceutically active agent is water insoluble or sparingly water soluble. In particular embodiments the pharmaceutically active agent is a water insoluble or sparingly water soluble macromolecule, peptide or protein. In particular embodiments, the pharmaceutically active agent is water-in-oil soluble. In particular embodiments the pharmaceutically active agent is a water-in-oil soluble macromolecule, peptide or protein.

Typically the functional oil comprises, or consists of, a vitamin oil. The functional oil may comprise, or consist of, a vitamin E oil. The functional oil may comprise, or consist of, one or more tocotrienols. The functional oil may comprise, or consist of, one or more tocopherols.

The at least one surfactant may be natural or synthetic. Typically the at least one surfactant is selected from the group consisting of a monoglyceride, lecithin, polyethylene glycol, propylene glycol, a glycerol derivative, a sugar alcohol, polyethoxylated castor oil, polyoxyethylene sorbitan monolaurate, polyoxyethylene sorbitan monoleate, tocopheryl polyethylene glycol succinate, a polyethoxylated sorbitan ester, a sorbitan ester, a triglyceride, a gelatin, a protein, a gel formulation, and mixtures thereof. In one embodiment the at least one surfactant is polyethylene glycol. In certain embodiments the at least one surfactant is polyethylene glycol 200.

Typically, the at least one active agent is selected from the group consisting of a hormone, a hormone analogue, a hormone mimetic, a glucagon-like peptide-1 agonist, a cytokine, an interleukin, interferon, a vaccine, an antibody, an allergen, a scaffold protein, and an oil soluble plant extract, and combinations thereof.

The hormone, hormone analogue or hormone mimetic may be a peptide hormone, peptide hormone analogue or peptide hormone mimetic. In exemplary embodiments, the peptide hormone is insulin, an incretin mimetic (glucose-dependent insulinotropic peptide, GIP; and glucagon-like peptide-1, GLP-1) or glucagon. In an exemplary embodiment, the peptide hormone mimetic is an incretin. In an exemplary embodiment, the incretin is a GLP-1 agonist. In an exemplary embodiment, the GLP-1 agonist is liraglutide. In one embodiment, the GLP-1 agonist is exenatide. In one embodiment, the peptide hormone analogue is insulin aspart or insulin glargine.

In exemplary embodiments, the delivery system comprises insulin, or an analogue or mimetic thereof, and a GLP-1 agonist. The GLP-1 agonist may be exenatide. The GLP-1 agonist may be liraglutide.

In one embodiment, the delivery system comprises insulin aspart and/or insulin glargine, and a GLP-1 agonist. The GLP-1 agonist may be exenatide or liraglutide.

In particular exemplary embodiments the delivery system is in the form of a spray, drops, or is absorbed onto a solid carrier.

The delivery system may further comprise at least one additional oil or oil mixture. The at least one additional oil or oil mixture may be present as, for example, a diluent, carrier, co-solvent flavouring agent or taste masking agent. The at least one additional oil or oil mixture may be an essential oil. The essential oil may be selected from the group consisting of camphor oil, menthol, eucalyptus oil, orange oil, mandarin oil, peppermint oil, clove oil, thymol, coconut oil, chlorbutol, arachis oil, sesame oil, lemon oil, spearmint oil, citronella oil, geranium oil, and combinations thereof.

A second aspect of the present disclosure provides a composition formulated for the delivery of pharmaceutically active agents via the sublingual and/or buccal mucosa, wherein the composition comprises at least one functional oil; at least one surfactant; and at least one pharmaceutically active agent. Exemplary active agents, functional oils and surfactants are as described above in relation to the first aspect. The composition may further comprise at least one additional oil, or oil mixture, as described herein.

A third aspect of the present disclosure provides a method for treating diabetes, for regulating blood glucose levels and/or for preventing or treating hyperglycaemia in a subject, the method comprising sublingually and/or buccally administering to the subject an effective amount of a delivery system according to the first aspect or a composition according to the second aspect.

### BRIEF DESCRIPTION OF THE DRAWINGS

Embodiments of the present disclosure are described herein, by way of non-limiting example only, with reference to the accompanying drawings.

FIG. 1. Blood glucose levels in diabetic mice at 30 to 60 minute time intervals relative to the administration (0 mins) of either: 5 μg sublingually administered exenatide (SL-Exenatide); 100 IU/kg sublingually administered insulin aspart (SL-Insulin aspart in TPMCEu); sublingually administered 100 IU/kg insulin aspart plus 5 μg exenatide (SL-Insulin aspart+exenatide in TPMCEu); 1 IU/kg subcutaneously administered insulin aspart (SC-Insulin aspart); or sublingual oil placebo (SL-TPMCEu).

FIG. 2. Area under the curve analysis (AUC) of blood glucose levels from 0-240 min, derived from the results shown in FIG. 1. **=p<0.01, ***=p<0.001, ****=p<0.0001 versus sublingual oil placebo (vitamin E plus polyethylene glycol 200).

FIG. 3. Blood glucose levels in diabetic mice at 0 h, 1 h, 2 h, 4 h, 8 h, 20 h and following administration of either: sublingual solid dosage formulation placebo (small circles): 1 IU/kg subcutaneously administered insulin glargine (diamonds); 10 IU/kg sublingually administered insulin glargine (large circles); and 50 IU/kg sublingually administered insulin glargine (squares).

FIG. 4. Blood glucose levels in diabetic mice at −30 min, 0 min, 30 min, 60 min, 120 min, 240 min, 480 min and 720 min relative to the administration (0 mins) of either: sublingual oil formulation placebo (Group 1: CSSR6b vehicle); 5 μg/mouse sublingually administered exenatide (Group 2: SL exenatide); sublingually administered 1 IU/kg insulin aspart+1 IU/kg insulin glargine+5 μg/mouse exenatide in CSSR6b (Group 3: SL insulin aspart 1 IU/kg+insulin glargine 1 IU/kg+exenatide 5 μg); or subcutaneously administered 1 IU/kg insulin aspart+1 IU/kg insulin glargine+1 μg/mouse exenatide in saline (Group 4: SC insulin aspart 1 IU/kg and insulin glargine 1 IU/kg+1 μg exenatide).

FIG. 5. Area under the curve analysis (AUC) of blood glucose levels from 0-720 min, derived from the results shown in FIG. 4. ****=p<0.0001 versus Group 1.

FIG. 6. Blood glucose levels in diabetic mice at −30 min, 0 min, 30 min, 60 min, 120 min, 240 min, 480 min and 720 min relative to the administration (0 mins) of sublingual oil formulation placebo (ovals: SL-CSSR6b vehicle); sublingually administered 0.5 IU/kg insulin aspart+0.5 IU/kg insulin glargine+2 μg/mouse exenatide oil formulation (squares: SL insulin aspart 0.5 IU/kg+insulin glargine 0.5 IU/kg+exenatide 2 μg); sublingually administered 0.5 IU/kg insulin aspart+0.5 IU/kg insulin glargine+1 μg/mouse exenatide oil formulation (triangles: SL insulin aspart 0.5 IU/kg+insulin glargine 0.5 IU/kg+exenatide 1 μg); sublingually administered 1 IU/kg insulin aspart+1 IU/kg insulin glargine+0.5 μg/mouse exenatide oil formulation (inverted triangles: SL insulin aspart 1 IU/kg+insulin glargine 1 IU/kg+exenatide 0.5 μg); sublingually administered 0.5 IU/kg insulin aspart+0.5 IU/kg insulin glargine+0.1 μg/mouse exenatide oil formulation (diamonds: SL insulin aspart 0.5 IU/kg+insulin glargine 0.5 IU/kg+exenatide 0.1 μg); and subcutaneously administered 1 IU/kg insulin aspart+1 IU/kg insulin glargine+1 μg/mouse exenatide in saline (inverted triangles: SC insulin aspart 1 IU/kg+insulin glargine 1 IU/kg+1 μg exenatide in saline).

FIG. 7. Area under the curve analysis (AUC) of blood glucose levels from 0-720 min, derived from the results shown in FIG. 6. *=p<0.05, ****=p<0.0001 versus Group 1.

FIG. 8. Blood glucose levels in DIO mice at −60 min, 0 min, 20 min, 30 min, 40 min, 60 min, 120 min and 240 min relative to the administration of sublingual water formulation (SL-water); sublingually administered 5 μg exenatide oil formulation (SL-Exenatide (5 μg/mouse)+CSSR3 TPPM 30/70 PH4.5); sublingually administered 10 exenatide oil formulation (SL-Exenatide (10 μg/mouse)+CSSR3 TPPM 30/70 PH4.5); and subcutaneously administered 1 μg exenatide saline formulation (SC-Exenatide 1 μg/mouse).

FIG. 9. Area under the curve analysis (AUC) of blood glucose levels from 0-720 min, derived from the results shown in FIG. 8. *=p<0.05, ****=p<0.0001 versus Group 1.

FIG. 10. Blood glucose levels in diabetic mice at −30 min, 0 min, 30 min, 60 min, 120 min, 240 min, 480 min and 720 min relative to the administration (0 mins) of sublingual oil formulation placebo (circles: SL-CSSR6d); subcutaneously administered 50 μg liraglutide in water (triangles: liraglutide SC (dd water) 50 μg); sublingually administered 150 μg liraglutide in oil formulation (inverted triangles: liraglutide SL (CSSR6d) 150 μg); sublingually administered 150 μg liraglutide in oil formulation (squares: liraglutide SL (CSSR6d3) 150 μg); and sublingually administered 20 IU/kg insulin glargine and 20 IU/kg insulin aspart in oil formulation (circles: insulin glargine 20 IU/kg and insulin aspart 20 IU/kg-SL (CSSR6d)).

FIG. 11. Area under the curve analysis (AUC) of blood glucose levels from 0-720 min, derived from the results shown in FIG. 10. ****=p<0.0001 versus Group 1.

FIG. 12. Cₘₐₓ and AUC analysis of blood resveratrol levels in rats administered sublingual resveratrol in oil formulation (Group 1: SL-resveratrol 10 mg in oil formulation); or oral administered resveratrol in sugar syrup (Group 2: OP resveratrol 10 mg in sugar syrup).

### DETAILED DESCRIPTION OF THE EMBODIMENTS

It will be understood that the invention disclosed and defined in this specification extends to all alternative combinations of two or more of the individual features mentioned or evident from the text or drawings. All of these different combinations constitute various alternative aspects of the invention.

Unless defined otherwise, all technical and scientific terms used herein have the same meaning as commonly understood by those of ordinary skill in the art to which the disclosure belongs. Although any methods and materials similar or equivalent to those described herein can be used in the practice or testing of the present disclosure, typical methods and materials are described.

Throughout this specification and the claims which follow, unless the context requires otherwise, the word “comprise”, and variations such as “comprises” or “comprising”, will be understood to imply the inclusion of a stated element, integer or step, or group of elements, integers or steps, but not the exclusion of any other element, integer or step, or group of elements, integers or steps.

The articles “a” and “an” are used herein to refer to one or to more than one (i.e., to at least one) of the grammatical object of the article. By way of example, “an element” means one element or more than one element.

In the context of this specification, the term “about” is understood to refer to a range of numbers that a person of skill in the art would consider equivalent to the recited value in the context of achieving the same function or result.

The term “subject” as used herein refers to any mammal, including, but not limited to, livestock and other farm animals (such as cattle, goats, sheep, horses, pigs and chickens), performance animals (such as racehorses), companion animals (such as cats and dogs), laboratory test animals and humans. Typically the subject is a human.

As used herein the terms “treating”, “treatment”, “reduce”, “reducing”, “prevent”, “preventing” and “prevention” and the like refer to any and all applications which remedy, or otherwise hinder, retard, or reverse the progression of, an infection or disease or at least one symptom of an infection or disease, including reducing the severity of an infection or disease. Thus, the terms “treat”, “treating”, “treatment”, do not necessarily imply that a subject is treated until complete elimination of the infection or recovery from a disease. Similarly, the terms “prevent”, “preventing”, “prevention” and the like refer to any and all applications that prevent the establishment of an infection or disease or otherwise delay the onset of an infection or disease.

The term “optionally” is used herein to mean that the subsequently described feature may or may not be present or that the subsequently described event or circumstance may or may not occur. Hence the specification will be understood to include and encompass embodiments in which the feature is present and embodiments in which the feature is not present, and embodiments in which the event or circumstance occurs as well as embodiments in which it does not.

As used herein the terms “effective amount” and “effective dose” include within their meaning a non-toxic but sufficient amount or dose of a pharmaceutically active agent to provide the desired effect. The exact amount or dose required will vary from subject to subject depending on factors such as the species being treated, the age and general condition of the subject, the severity of the condition being treated, the particular active agent(s) being administered and the mode of administration and so forth. Thus, it is not possible to specify an exact “effective amount” or “effective dose”. However, for any given case, an appropriate “effective amount” or “effective dose” may be determined by one of ordinary skill in the art using only routine experimentation.

As used herein the term “delivery system” refers to a composition comprising a formulation according to the present disclosure which is particularly adapted for delivery of pharmaceutically active agents via the sublingual and/or buccal mucosa.

As used herein, the terms “sublingual” and “buccal” refer to the sublingual and buccal mucosal regions and linings in the mouth. Thus, delivery or administration of delivery systems and compositions according to the present disclosure via the sublingual and/or buccal mucosa are distinguished from ingestion or other means of delivery via the gastrointestinal tract.

As used herein the term “oil” refers to a non-polar chemical substance that is hydrophobic and lipophilic. Those skilled in the art will appreciate that an oil may be a natural oil that it is animal, plant or petrochemical in origin; may be derived from or extracted from a natural oil via a physical or chemical process; or may be a synthetic oil.

As used herein the term “functional oil” refers to an oil that has, in the context of delivery systems, formulations, compositions and methods of the present disclosure, an action beyond its nutritional value. For example, Vitamin E has nutritional value as a vitamin, however in the formulations according to the present disclosure Vitamin E oil also acts as an oil delivery base. Those skilled in the art will appreciate that functional oils are not limited to vitamins but include any oil which has an action beyond its nutritional value.

As used herein the term “extract” refers to an active preparation derived from one or more plants or a synthetic version thereof. In the context of the specification by “active” it is meant that the extract is capable of producing a desired therapeutic benefit. An extract is obtained by a process of “extraction” which will be understood by those skilled in the art as, in general terms, comprising treating plant material with a solvent, a liquid, or a supercritical fluid to dissolve the active preparation and separate the same from residual unwanted plant material. An extract may be in liquid form (for example as a decoction, solution, infusion or tincture) or solid form (for example as a powder or granules). An extract may comprise a single active agent or a combination of active agents.

In the context of this specification, the term “plant” refers to any living organism that grows in the earth, including but not limited to, trees, shrubs, flowers, bushes, herbs, grasses, ferns, and mosses and any plant material derived therefrom (for example, fruit, fruit skin, leaves, seed, bark, roots, stems and the like).

The present inventors have developed compositions specifically adapted for delivery of pharmaceutically active ingredients via the sublingual and buccal mucosa, in particular for the delivery of those pharmaceutically active ingredients that are water insoluble or poorly water-soluble. Accordingly, embodiments of the present disclosure provide compositions and sublingual and/or buccal transmucosal delivery systems comprising at least one functional oil; at least one surfactant; and at least one pharmaceutically active agent, typically wherein the pharmaceutically active agent is water insoluble or poorly water-soluble. The pharmaceutically active agent may be water-in-oil soluble. In some embodiments the delivery system further comprises at least one additional oil, such as an essential oil.

The present disclosure beneficially provides delivery of pharmaceutically active agents via the sublingual and buccal mucosa, ensuring rapid delivery and absorption, avoiding the acidic environment of the stomach and enzymatic digestion in the gastrointestinal tract, and avoiding the need to break the skin, as with, for example an injection. Whilst being advantageous for the administration of any pharmaceutically active agent, delivery systems of the present disclosure will find particular use with pharmaceutically active agents which are water insoluble, sparingly soluble or water-in-oil soluble macromolecules, peptides and proteins.

Without wishing to be bound by theory or mode of action, the present inventors believe that the formulation of the present invention results in the active agent being absorbed into the lymphatic system, as opposed to the blood. For example, it has been demonstrated in the literature (D'Alessio, D. et al (2007) Am J Physiol Requl Integr Comp Physiol, 293(6): R2163-9) that the post-prandial concentration of large molecules, such as GLP-1, is substantially higher in lymph than in blood plasma. Lymph is a protective environment for such molecules, and allows them to bypass phase II metabolism, which prolongs their half-life in the body. In addition, absorption through oral mucosa bypasses first-pass metabolism in the liver, which further improves the half-life of the active.

The delivery systems and compositions of the present disclosure comprise at least one functional oil. The at least one functional oil may be one or more of vitamin oil, palm oil, rice bran oil, marine oil (e.g. fish oil or krill oil), citrus oil, flax oil, castor oil, sunflower oil, olive oil, soybean oil, corn oil, walnut oil, peanut oil, almond oil, or an oil derived from rice, wheat, barley, rye, oats, saw palmetto or annatto. The functional oil may comprise, or consist of, a marine oil. The functional oil may comprise, or consist of, one or more omega-3 fatty acids. The omega-3 fatty acid may be selected from alpha-linolenic acid (ALA), eicosapentaenoic acid (EPA) and docosahexaenoic acid (DHA).

The functional oil may comprise, or consist of, a vitamin oil, palm oil, rice bran oil, sunflower oil, olive oil, soybean oil, corn oil, walnut oil, peanut oil, almond oil, or an oil derived from rice, wheat, barley, rye, oats, saw palmetto or annatto, or mixtures of these oils. The functional oil may comprise, or consist of, a vitamin oil. The functional oil may comprise, or consist of, vitamin E oil. The Vitamin E oil may be a natural oil that it is derived from an animal, or plant source. For example, the Vitamin E oil may be derived from a vegetable oil, such as soybean oil, wherein the Vitamin E oil is extracted via a physical or chemical process. The Vitamin E oil may be a synthetic oil.

The functional oil may comprise, or consist of, one or more tocotrienols. The tocotrienol may be selected from one or more compounds of formula (I):

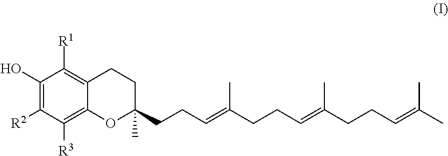

wherein:

R¹, R² and R³ are independently selected from H and CH₃.

R¹, R² and R³ may be CH₃. R¹ and R³ may be CH₃, and R² may be H. R¹ may be H and R² and R³ may be CH₃. R¹ and R² may be H, and R³ may be CH₃. The tocotrienol may be α-tocotrienol. The tocotrienol may be β-tocotrienol. The tocotrienol may be γ-tocotrienol. The tocotrienol may be δ-tocotrienol.

The functional oil may comprise, or consist of, one or more tocopherols. The tocopherol may be selected from one or more compounds of formula (II):

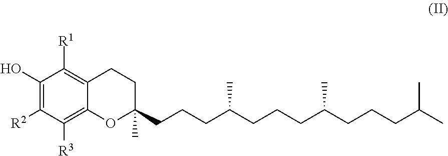

wherein:

R¹, R² and R³ are independently selected from H and CH₃.

R¹, R² and R³ may be CH₃. R¹ and R³ may be CH₃, and R² may be H.

R¹ may be H and R² and R³ may be CH₃. R¹ and R² may be H, and R³ may be CH₃. The tocopherol may be α-tocopherol. The tocopherol may be β-tocopherol. The tocopherol may be γ-tocopherol. The tocopherol may be δ-tocopherol.

The functional oil may be tocopherol-free i.e. tocopherol is present in an amount below the measurable limit of, for example, α-tocopherol. The functional oil may be enriched in tocotrienols. That is, the functional oil may have, for example, greater than 50%, greater than 55%, greater than 60%, greater than 65%, or greater than 70%, or greater than 75% (w/w) tocotrienols. The functional oil may be enriched in α-tocotrienol, β-tocotrienol, γ-tocotrienol and/or δ-tocotrienol. For example, the oil may have greater than 50%, greater than 55%, greater than 60%, greater than 65%, greater than 70%, greater than 75%, greater than 80%, greater than 85%, greater than 90%, or greater than 95% (w/w) of one or more of α-tocotrienol, β-tocotrienol, γ-tocotrienol and δ-tocotrienol. The functional oil may comprise, or consist of, a mixture of one or more tocotrienols and one or more tocopherols.

The delivery systems and compositions of the present disclosure comprise at least one surfactant. The at least one surfactant may include but is not limited to a monoglyceride, lecithin, polyethylene glycol, propylene glycol, a glycerol derivative, a sugar alcohol, polyethoxylated castor oil, polyoxyethylene sorbitan monolaurate, polyoxyethylene sorbitan monoleate, tocopheryl polyethylene glycol succinate, a polyethoxylated sorbitan ester, a sorbitan ester, a triglyceride and mixtures thereof. In some embodiments the at least on surfactant is polyethylene glycol. The polyethylene glycol may have a molecular weight of from 200 to 8000. In exemplary embodiments the polyethylene glycol has a molecular weight of 200, 400, 300, 600 or 1000. In a particular exemplary embodiment, the polyethylene glycol has a molecular weight of 200.

The delivery systems and compositions of the present disclosure comprise at least one pharmaceutically active agent. Typically the active agent is a water insoluble or sparingly water soluble macromolecule, peptide or protein. The active agent may be a water-in-oil soluble macromolecule, peptide or protein. Such macromolecules, peptides and proteins include, but are not limited to hormones, hormone analogues, hormone mimetics, glucagon-like peptide-1 agonists, cytokines, interleukins, interferons, vaccines, antibodies, allergens, scaffold proteins, and oil soluble plant extracts. Analogues of such macromolecules, peptides and proteins are also contemplated. In the present context, an analogue means a derivative of a macromolecule, peptide or protein that retains substantially the same function and physiological properties as the macromolecule, peptide or protein from which it is derived. In the case of peptides and proteins, such derivatives may comprise the addition, deletion or substitution of one or more amino acids.

In some embodiments the active agent is a hormone. The hormone may be selected from the group consisting of testosterone, insulin, progesterone and estrogen. However a person skilled in the art will appreciate that other hormones may also be used.

In particular embodiments the hormone, hormone analogue or hormone mimetic is a peptide hormone, peptide hormone analogue or peptide hormone mimetic. Typically the peptide hormone is a human peptide hormone. The peptide hormone may comprise the native human sequence of the mature hormone or a derivative, variant or homologue thereof. Precursor, recombinant or modified forms of the hormone may also be used. By way of example only, the peptide hormone may be selected from insulin, an incretin (glucose-dependent insulinotropic peptide, GIP; and glucagon-like peptide-1, GLP-1), glucagon, somatostatin, oxytocin, vasopressin, leptin, prolactin, ACTH and cholecystokinin. By way of example only, the peptide hormone mimetic may be selected from exenatide, octreotide and liraglutide. In an exemplary embodiment, the peptide hormone analogue is insulin aspart or insulin glargine.

Other peptides that may be employed as active agents include metabolic agents (such as semaglutide, pramlintide, peptide YY, dulaglutide, lixisenatide and albiglutide), as well as other agents that act on the endocrine system but are not metabolics (such as lanreotide, sincalide, octreotide, degarelix and tesamorelin, antifungal agents (such as anidulafungin, micafungin, caspofungin, dalbavancin, oritavancin and telavancin), antivirals (such as enfurvitide, telaprevir and boceprevir), anti-cancer agents (such as everolimus, midostaurin, eribulin, romidepsin, carfilzomib, oprozomib, ixazomib, leuprolide, goserelin, bortezomib and mifamurtide), agents for gastroenterological disorders (such as plecanatide, teduglutide and linaclotide), agents for reproductive health (such as cetrorelix, teriparatide, gonadotropin-releasing hormone (GnRH), and gondaotropins LH and FSH), agents for cardiovascular disorders (such as nesiritide, icatibant, bivalirudin and eptifibatide), agents for CNS disorders (such as glatiramer), agents for hematological disorders (such as icatibant and ecallantide), and analgesics (such as ziconotide).

In some embodiments the active agent comprises insulin, or an analogue or mimetic thereof. The insulin, insulin analogue or mimetic may be a fast acting form (such as, for example, insulin aspart) or a long acting form (such as, for example, insulin glargine). A variety of other forms, analogues and mimetics of insulin are known to those skilled in the art and are contemplated within the scope of the present disclosure.

In some embodiments the active agent is a glucagon-like peptide-1 agonist. The GLP-1 agonist may be selected from the group consisting of exenatide, liraglutide, lixisenatide, albiglutide, duglutide, semaglutide, and analogues thereof. The GLP-1 agonist may be exenatide, or an analogue thereof. One exemplary analogue is AC3174 in which the methionine residue at position 14 of exenatide is replaced by a leucine residue. The GLP-1 agonist may be liraglutide, or an analogue thereof.

The delivery system may comprise a combination of active agents. In one embodiment, the delivery system comprises insulin aspart and/or insulin glargine, and a GLP-1 agonist. The GLP-1 agonist may be exenatide or liraglutide.

In some embodiments the active agent is a cytokine. Typically the cytokine is a human cytokine. The cytokine may comprise the native human sequence of the mature cytokine or a derivative, variant or homologue thereof. Precursor, recombinant or modified forms of the cytokine may also be used. The cytokine may be selected from the group consisting of a chemokine, an interferon, an interleukin and a tumour necrosis factor. Interferons (IFNs) include but are not limited to, IFNγ, IFN-1β, and IFNα. Interleukins include but are not limited to IL-1, IL-2, IL-113, IL-6, IL-7, IL-8, IL-12, IL-15, IL-18, IL-21, and granulocyte-macrophage colony-stimulating factor (GM-CSF). The tumour necrosis factor (TNF) may be TNFα. Those skilled in the art will appreciate that other cytokines may also be employed and the scope of the present disclosure is not limited by reference to any particular cytokine.

Other exemplary pharmaceutically active agents include, for example, erythropoietin and cannabinoids such as cannabidiol and cannabidiol extracts, flax seed extracts, alpha-glycerophosphocholine (Alpha-GPC or α-GPC), pyrroloquinoline quinone (PQQ), pterostilbene, resveratrol, thymoquinone, dihydroquercetin (DHQ), oleoylethanolamide (OEA), citicoline, magnesium L-threonate, palmitoylethanolamide (PEA), phosphatidylserine (PS), bacopa extracts, isoflavones, osthole, beta-1,3-glucan, lutein, lycopene, octanosol, policosanol, co-enzyme Q10, fat-soluble vitamins, oxytoxin, and carboxytocin.

However those skilled in the art will appreciate that other agents may also be employed and the scope of the present disclosure is not limited by reference to any particular agent.

The delivery systems and compositions of the present disclosure may further comprise at least one additional oil, or oil mixture. The additional oil or oil mixture may act as a diluent, carrier, co-solvent, flavour, and/or taste masking agent. The oil or oil mixture may increase the stability of the delivery system. The oil or oil mixture may comprise a natural oil in that it is animal, plant or petrochemical in origin; may be derived from or extracted from a natural oil via a physical or chemical process; or may be a synthetic oil. The oil may be an essential oil. Examples of suitable oils and oil mixtures include essential oils, such as camphor oil, menthol, eucalyptus oil, orange oil, mandarin oil, peppermint oil, clove oil, coconut oil, chlorbutol, arachis oil, lemon oil, spearmint oil, citronella oil, geranium oil, and thymol. Other examples of suitable additional oils include sunflower oil, soybean oil, canola oil, olive oil, corn oil, peanut oil, groundnut oil, rice bran oil, cottonseed oil, flax seed oil, palm oil, palm kernel oil, safflower oil, soybean oil, sesame oil, amaranth oil, linseed oil, argan oil, grapeseed oil, cranberry seed oil, hazelnut oil, hemp oil, jojoba oil, macadamia oil, mustard oil, neem oil, orange oil, rapeseed oil, avocado oil, almond oil, sweet almond oil, cashew oil, castor oil, vegetable oil, walnut oil, wheatgerm oil, kukui nut oil, tamuna oil, aloe vera oil, apricot kernel oil, borage oil (from, for example Borago officionalis), camellia oil (from, for example, Camellia oleifera), cocoa butter oil, rosehip see oil, fish oils, ethyl oleate, ethyl linoleate, saturated fatty acids (such as, but not limited to, caproic acid, caprylic acid, capric acid, lauric acid, valeric acid, myristic acid, palmitic acid, stearic acid, arachidic acid), medium chain triglycerides, omega-3 fatty acids (such as, but not limited to, hexadecatrienoic acid, alpha-linolenic acid, stearidonic acid, ecosatrienoic acid, eicosapentaenoic acid, heneicosapentanoic acid, docosapentanoic acid, docosahexanoic acid, tetracosapentaenoic acid, tetracosahexanenoic acid), omega-6 fatty acids (such as, but not limited to, linoleic acid, gamma-linolenic acid, eicosadienoic acid, dihomo-gamma-linolenic acid, arachidonic acid, docosadienoic acid, adrenic acid, docosapentaenoic acid, tetracosatetraenoic acid, tetracosapentaenoic acid), and/or omega-9 fatty acids (such as, but not limited to, oleic acid, eicosenoic acid, mead acid, erucic acid, nervonic acid). In particular embodiments the oil or oil mixture is an essential and/or a volatile oil or oil mixture.

Transmucosal delivery systems and compositions according to the present disclosure may be in any form suitable for delivery of active agents via the sublingual and/or buccal mucosa, including for example drops, sprays, pumps, gels, foams and quick dissolve tablets. The skilled artisan will appreciate that the transmucosal delivery systems and compositions are not so limited and that any transmucosal formulations may be employed.

In particular embodiments the formulation of the delivery system or composition is such that it provides oil drops or oil spray being suitable for administration sublingually and/or buccally. Oil drops and oil sprays and other forms of the delivery systems and compositions of the present disclosure may be administered using any suitable conventional administration means (for example via a pipette dropper, or via a pump action or pressurized administration vessel such as an aerosol spray). In particular embodiments the administration means may provide metered doses of the composition. While oil drops and oil sprays for delivery sublingually or buccally via the mucosa are particularly advantageous forms of the delivery system, those skilled in the art will appreciate that the delivery system may be delivered in other forms.

In other embodiments the delivery system or composition can be absorbed onto solid carriers, such as but not limited to tablets, powders, granules, or beads and delivered sublingually and/or buccally. In general, suitable solid carriers may be prepared according to methods that are known to those of ordinary skill in the art and may include a pharmaceutically acceptable diluent, adjuvant and/or excipient. The diluents, adjuvants and excipients must be “acceptable” in terms of being compatible with the other ingredients of the composition, and not deleterious to the recipient thereof.

A broad range of processes for the preparation of solid carriers suitable for sublingual and buccal administration are well known to those skilled in the art and are contemplated by the present disclosure. For example, suitable solid carriers may be prepared by processes including freeze drying under vacuum, supercritical fluid drying, spray drying using heat, and fluid bed spray drying. Of particular application in the context of particular embodiments of the present disclosure is a process involving microencapsulation whereby the delivery system is absorbed onto granules, tablets or microparticles. One particularly suitable process is disclosed in International Patent Application Publication No. WO 02/058735 (the disclosure of which is incorporated herein in its entirety by reference). Also known in the art are means for the preparation of compositions incorporating an effervescent agent as a penetration enhancer to increase the permeability of the active agent across the sublingual and buccal mucosa (see for example U.S. Pat. No. 6,974,590, the disclosure of which is incorporated herein in its entirety by reference).

Those skilled in the art will readily appreciate that a number of suitable processes and techniques exist for the manufacture of suitable solid carriers in accordance with the present disclosure and that the disclosure is not limited by reference to any one particular process or technique.

The therapeutically effective dose level of a delivery system or composition of the present disclosure for any particular patient will depend upon a variety of factors including any one or more of: the nature of condition being treated and the stage of the condition; the activity of the active agent employed; the composition employed; the age, body weight, general health, sex and diet of the patient; the time of administration; the rate of sequestration of compounds; the duration of the treatment; the active agent used in combination or coincidental with the treatment, together with other related factors well known in medicine. One skilled in the art would be able, by routine experimentation, to determine an effective, non-toxic dosage. These will most often be determined on a case-by-case basis.

By way of example, for sublingual or buccal administration of exenatide, individual doses of, for example, about 0.1 μg to about 5 mg may be used. Typically the dose employed in accordance with the present disclosure is between about 0.5 μg and about 1 mg or between about 1 μg and about 600 μg. The dose may be in the order of about 5 μg, 10 μg, 15 μg, 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 105 μg, 110 μg, 115 μg, 120 μg, 125 μg, 130 μg, 135 μg, 140 μg, 145 μg, 150 μg, 200 μg, 250 μg, 300 μg, 350 μg, 400 μg, 450 μg, 500 μg, 550 μg or 600 μg. In particular embodiments the dose may be about 100 μg to about 200 μg, optionally administered two or three times daily, and optionally administered about 30-120 minutes (typically 60 minutes) before a meal.

By way of example, for sublingual or buccal administration of liraglutide, individual doses of, for example, about 100 μg to about 600 mg may be used. Typically the dose employed in accordance with the present disclosure is between about 1 mg and about 50 mg. The dose may be in the order of about 1 mg, 2 mg, 3 mg, 4 mg, 5 mg, 6 mg, 7 mg, 8 mg, 9 mg, 10 mg, 11 mg, 12, mg, 13 mg, 14 mg, 15 mg, 16 mg, 17 mg, 18 mg, 19 mg or 20 mg. In particular embodiments the dose may be about 5 mg to about 20 mg optionally administered two or three times daily, and optionally administered about 30-120 minutes (typically 60 minutes) before a meal.

By way of example, for sublingual or buccal administration of insulin, for example insulin aspart or insulin glargine, individual doses of, for example, about 0.1 IU/kg to about 1000 IU/kg mg may be used. Typically the dose employed in accordance with the present disclosure is between about 10 IU/kg and about 600 IU/kg. The dose may be in the order of about 15 IU/kg, 20 IU/kg, 25 IU/kg, 30 IU/kg, 35 IU/kg, 40 IU/kg, 45 IU/kg, 50 IU/kg, 55 IU/kg, 60 IU/kg, 65 IU/kg, 70 IU/kg, 75 IU/kg, 80 IU/kg, 85 IU/kg, 90 IU/kg, 95 IU/kg, 100 IU/kg, 105 IU/kg, 110 IU/kg, 115 IU/kg, 120 IU/kg, 125 IU/kg, 130 IU/kg, 135 IU/kg, 140 IU/kg, 145 IU/kg, 150 IU/kg, 200 IU/kg, 250 IU/kg, 300 IU/kg, 350 IU/kg, 400 IU/kg, 450 IU/kg, 500 IU/kg, 550 IU/kg, or 600 IU/kg. In particular embodiments the dose may be about 50 IU/kg to about 500 IU/kg, optionally administered two or three times daily, and optionally administered about 30-120 minutes (typically 60 minutes) before a meal.

By way of example, for sublingual or buccal administration of a combination of insulin aspart and insulin glargine, individual doses of, for example, about 0.1 IU/kg to about 1000 IU/kg mg (of total insulin i.e. insulin aspart plus insulin glargine) may be used. Typically the dose employed in accordance with the present disclosure is between about 10 IU/kg and about 600 IU/kg. The dose may be in the order of about 15 IU/kg, 20 IU/kg, 25 IU/kg, 30 IU/kg, 35 IU/kg, 40 IU/kg, 45 IU/kg, 50 IU/kg, 55 IU/kg, 60 IU/kg, 65 IU/kg, 70 IU/kg, 75 IU/kg, 80 IU/kg, 85 IU/kg, 90 IU/kg, 95 IU/kg, 100 IU/kg, 105 IU/kg, 110 IU/kg, 115 IU/kg, 120 IU/kg, 125 IU/kg, 130 IU/kg, 135 IU/kg, 140 IU/kg, 145 IU/kg, 150 IU/kg, 200 IU/kg, 250 IU/kg, 300 IU/kg, 350 IU/kg, 400 IU/kg, 450 IU/kg, 500 IU/kg, 550 IU/kg, or 600 IU/kg. In particular embodiments the dose may be about 10 IU/kg to about 50 IU/kg, optionally administered two or three times daily, and optionally administered about 30-120 minutes (typically 60 minutes) before a meal.

By way of example, for sublingual or buccal administration of a combination of insulin aspart, insulin glargine and exenatide, individual doses as set out above in respect of insulin and exenatide (e.g. about 0.1 IU/kg to about 1000 IU/kg of total insulin, and about 0.1 μg to about 5 mg of exenatide) may be used.

It will also be apparent to one of ordinary skill in the art that the optimal quantity and spacing of individual dosages will be determined by the nature and extent of the condition being treated, the active agent, the form, route and site of administration, and the nature of the particular individual being treated. Also, such optimum conditions can be determined by conventional techniques known to those skilled in the art. For example, a subject may be administered the desired daily dose in a single unit dosage form once per day, or in two unit dosage forms administered twice a day.

It will also be apparent to one of ordinary skill in the art that the optimal course of treatment, such as, the number of doses of the composition given per day for a defined number of days, can be ascertained by those skilled in the art using conventional course of treatment determination tests.

Embodiments of the present disclosure contemplate the administration of one or more additional compounds or molecules together with the pharmaceutically active agent(s). Such additional compounds or molecules may be formulated together with the active agent(s) in a single delivery system or composition.

Delivery systems and compositions described herein comprising a glucagon-like peptide-1 agonist, for example exenatide or liraglutide, and/or insulin or a insulin analogue or mimetic, may find particular use in the treatment of diabetes; for regulating blood glucose levels; and/or for preventing or treating hyperglycaemia particularly in those individuals who are unable or unwilling to tolerate administration of insulin via the traditional injection methods or where compliance with such traditional injection methods is otherwise low.

Accordingly, the present invention also relates to a method of treating diabetes, for regulating blood glucose levels and/or for preventing or treating hyperglycaemia in a subject the method comprising sublingually and/or buccally administering to the subject an effective amount of a delivery system comprising: at least one functional oil; at least one surfactant; and at least one pharmaceutically active agent. The present invention also relates to the use of a delivery system comprising: at least one functional oil; at least one surfactant; and at least one pharmaceutically active agent, for treating diabetes, for regulating blood glucose levels and/or for preventing or treating hyperglycaemia. The present invention also provides a pharmaceutical composition for use in treating diabetes, for regulating blood glucose levels and/or for preventing or treating hyperglycaemia, in any of the embodiments described in the specification. The present invention also relates to the use of a delivery system comprising: at least one functional oil; at least one surfactant; and at least one pharmaceutically active agent, for the manufacture of a medicament for treating diabetes, for regulating blood glucose levels and/or for preventing or treating hyperglycaemia.

The present invention also relates to a delivery system comprising: at least one functional oil; at least one surfactant; and at least one pharmaceutically active agent, when used in a method of treating diabetes, of regulating blood glucose levels and/or of preventing or treating hyperglycaemia. The present invention also relates to a composition having at least one functional oil, at least one surfactant, and at least one an active ingredient for use in treating diabetes, for regulating blood glucose levels and/or for preventing or treating hyperglycaemia. The present invention also relates to the use of a pharmaceutical composition containing at least one functional oil; at least one surfactant; and at least one pharmaceutically active agent, in treating diabetes, for regulating blood glucose levels and/or for preventing or treating hyperglycaemia, such as described above.

The skilled artisan will appreciate that the delivery systems and compositions according to the present disclosure will find use in methods for managing, preventing and/or treating diseases or conditions in accordance with any and all indications to which the active agent(s) of choice finds use. The skilled artisan will appreciate that these active agents and others to which delivery systems and compositions of the present disclosure relate, are not limited to known treatments.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

The present disclosure will now be described with reference to the following specific examples, which should not be construed as in any way limiting the scope of the disclosure.

### EXAMPLES

The following examples are illustrative of the disclosure and should not be construed as limiting in any way the general nature of the disclosure of the description throughout this specification.

### Materials

Exenatide was obtained from BioLingus Technology Development (Hong Kong) Pty Ltd. Insulin aspart and insulin glargine were obtained from YiChang HEC Changjiang Pharmaceutical Company Ltd.

### Example 1—Sublingual Insulin Aspart Study

Insulin aspart (a fast acting insulin analogue), exenatide or a mixture of insulin aspart and exenatide was dissolved in an oil comprising 20% Vitamin E oil, 20% polyethylene glycol 200, 20% chlorbutol, and 40% menthol/eucalyptol diluent/flavouring oils (TPMCEu) and the mixture spun until homogenous.

Male diabetic mice (strain C57/BL6) were sourced from Shanghai SLAC Laboratory Animal Co. Ltd. (Shanghai, China). For the experiments described below, mice of approximately 60 g were used. 5 μL of the resulting oil mixture comprising Insulin Aspart was placed underneath the tongue of anesthetised diabetic mice. Blood glucose was measured at −30 min, 0 min, 30 min, 60 min, 120 min and 240 min.

Five groups of five mice were treated. The treatment groups were administered as follows: 1) 5 μg sublingually administered exenatide (SL-Exenatide); 2) 100 U/kg sublingually administered insulin aspart (SL-Insulin aspart in TPMCEu); and 3) sublingually administered 100 U/kg insulin aspart and 5 μg exenatide (SL-Insulin aspart and exenatide in TPMCEu). These were compared to 4) 1 IU/kg subcutaneously administered insulin aspart (SC-Insulin aspart) and 5) sublingual oil placebo (SL-TPMCEu).

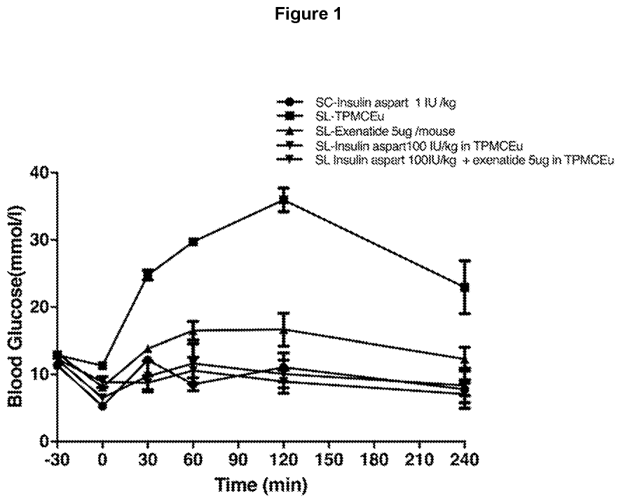

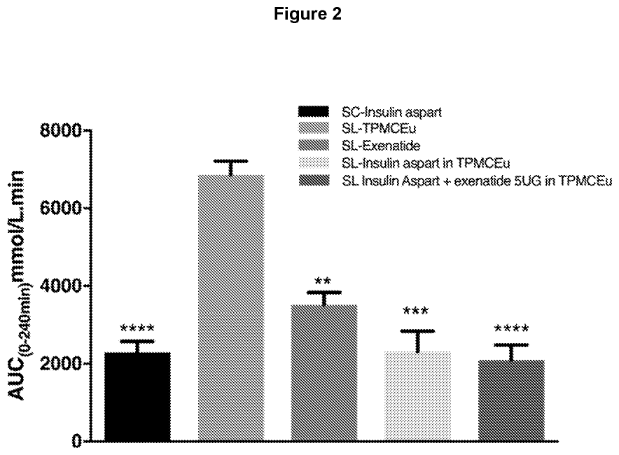

Blood glucose levels in each treatment group at −30 min, 0 min, 30 min, 60 min, 120 min and 240 min relative to the time of administration are shown in FIG. 1. Area under the curve analysis of blood glucose levels between 0 mins and 240 mins (AUC₀₋₂₄₀ₘᵢₙ) for each treatment group are shown in FIG. 2.

The results of these experiments demonstrate that sublingual delivery systems of insulin aspart according to the present disclosure, with or without exenatide, are comparable to subcutaneous delivery of insulin aspart.

### Example 2—Sublingual Insulin Glargine Study

Insulin glargine (a long acting insulin analogue) was dissolved in an oil comprising 20% Vitamin E oil, 20% polyethylene glycol 200, 20% chlorbutol, and 40% menthol/eucalyptol diluent/flavouring oils (TPMCEu), the mixture was spun until homogenous and absorbed into a solid dose formulation.

Male diabetic mice (strain C57/BL6) were sourced from Shanghai SLAC Laboratory Animal Co. Ltd. (Shanghai, China). One solid dosage formulation was placed underneath the tongue of anesthetised diabetic mice. Blood glucose was measured at 0 h, 1 h, 2 h, 4 h, 8 h, 20 h and 24 h.

Four groups of five mice were treated. The treatment groups were administered as follows: 1) sublingual solid dosage formulation placebo (Group 1: SL-CSSR 4; wherein “CSSR-4” means a solid formulation containing vitamin E, PEG 200 and flavouring oils). 2) 1 IU/kg subcutaneously administered insulin glargine (Group 2: SC-Insulin glargine 1 IU/kg); 3) 10 IU/kg sublingually administered insulin glargine (Group 3: SL-Insulin glargine 10 IU/kg); and 4) 50 IU/kg sublingually administered insulin glargine (Group 4: SL-Insulin glargine 50 IU/kg). For animal studies using solid formulations, tablets were dissolved in water to form a gel. The gel was then placed under the tongue of the mice instead of the tablet.

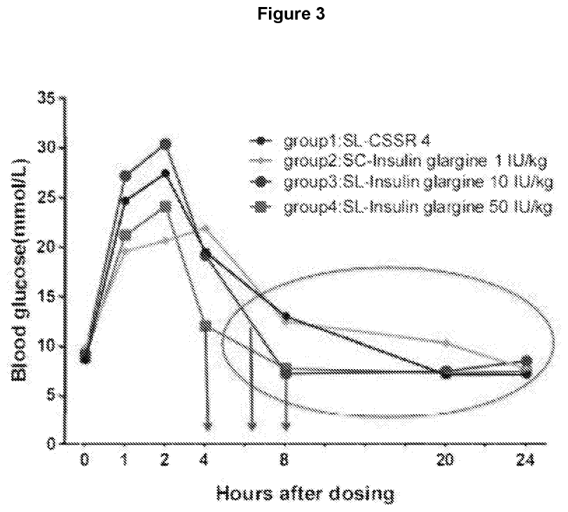

Blood glucose levels in each treatment group at 0 h, 1 h, 2 h, 4 h, 8 h, 20 h and 24 h relative to the time of administration are shown in FIG. 3.

The results of these experiments demonstrate that sublingual delivery systems of insulin glargine absorbed on solid dosage formulations according to the present disclosure are comparable to subcutaneous delivery of insulin glargine.

### Example 3—“InsulinPlus” Sublingual Insulin Aspart/Insulin Glargine/Exenatide Study 1

Insulin aspart, exenatide and/or insulin glargine was dissolved in an oil comprising 20% Vitamin E oil, 20% polyethylene glycol 200, 20% chlorbutol, and 40% menthol/eucalyptol diluent/flavouring oils (“CSSR6b”) and the mixture spun until homogenous.

Male diabetic mice (strain C57/BL6) were sourced from Shanghai SLAC Laboratory Animal Co. Ltd. (Shanghai, China). For the experiments described below, mice of approximately 50 g were used. 5 μL of the resulting oil mixture comprising the active agent(s) was placed underneath the tongue of anesthetised diabetic mice. Blood glucose was measured at −30 min, 0 min, 30 min, 60 min, 120 min, 240 min, 480 min and 720 min. The mice were allowed access to food after 4 h.

Four groups of five mice were treated. The treatment groups were administered as follows: 1) sublingual oil formulation placebo (Group 1: SL-CSSR6b vehicle); 2) 5 μg/mouse sublingually administered exenatide (Group 2: SL exenatide 5 μg/mouse); 3) sublingually administered 1 IU/kg insulin aspart+1 IU/kg insulin glargine+5 μg/mouse exenatide in CSSR6b (Group 3: SL insulin aspart 1 IU/kg+insulin glargine 1 IU/kg+exenatide); 4) subcutaneously administered 1 IU/kg insulin aspart+1 IU/kg insulin glargine+1 μg/mouse exenatide in saline (Group 4: SC insulin aspart 1 IU/kg and insulin glargine 1 IU/kg+1 μg exenatide).

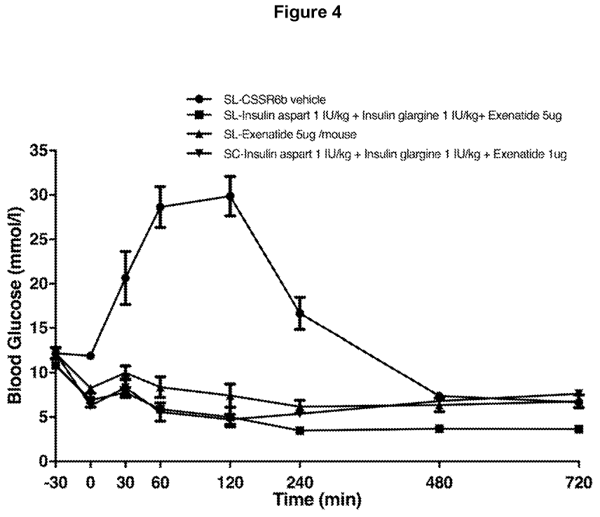

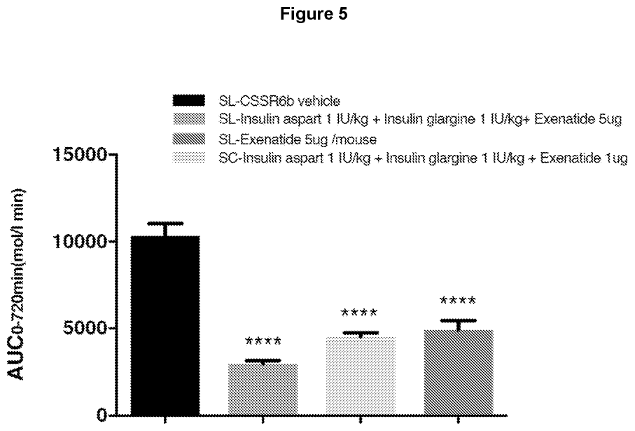

Blood glucose levels in each treatment group at −30 min, 0 min, 30 min, 60 min, 120 min, 240 min, 480 min and 720 min relative to the time of administration are shown in FIG. 4. Area under the curve analysis of blood glucose levels between 0-720 min (AUC₀₋₇₂₀ₘᵢₙ) for each treatment group are shown in FIG. 5.

The results of these experiments demonstrate that sublingual delivery systems of insulin aspart, insulin glargine and exenatide according to the present disclosure are comparable to subcutaneous delivery of insulin aspart and insulin glargine with or without exenatide.

### Example 4—“InsulinPlus” Sublingual Insulin Aspart/Insulin Glargine/Exenatide Study 2

Insulin aspart, exenatide and/or insulin glargine was dissolved in an oil comprising 20% Vitamin E oil, 20% polyethylene glycol 200, and 60% flavouring oils (“CSSR6d”), and the mixture spun until homogenous.

Male diabetic mice (strain C57/BL6) were sourced from Shanghai SLAC Laboratory Animal Co. Ltd. (Shanghai, China). For the experiments described below, mice of approximately 50 g were used. 5 μL of the resulting oil mixture comprising the active agent(s) was placed underneath the tongue of anesthetised diabetic mice. Blood glucose was measured at −30 min, 0 min, 30 min, 60 min, 120 min, 240 min, 480 min and 720 min. The mice were allowed access to food after 4 h.

Six groups of five mice were treated. The treatment groups were administered as follows: 1) sublingual oil formulation placebo (Group 1: SL-CSSR6d); 2) sublingually administered 0.5 IU/kg insulin aspart+0.5 IU/kg insulin glargine+2 μg/mouse exenatide oil formulation (Group 2: SL insulin aspart 0.5 IU/kg+insulin glargine 0.5 IU/kg+exenatide 2 μg); 3) sublingually administered 0.5 IU/kg insulin aspart+0.5 IU/kg insulin glargine+1 μg/mouse exenatide oil formulation (Group 3: SL insulin aspart 0.5 IU/kg+insulin glargine 0.5 IU/kg+exenatide 1 μg); 4) sublingually administered 1 IU/kg insulin aspart+1 IU/kg insulin glargine+0.5 μg/mouse exenatide oil formulation (Group 4: SL insulin aspart 1 IU/kg+insulin glargine 1 IU/kg+exenatide 0.5 μg); 5) sublingually administered 0.5 IU/kg insulin aspart+0.5 IU/kg insulin glargine+0.1 μg/mouse exenatide oil formulation (Group 5: SL insulin aspart 0.5 IU/kg+insulin glargine 0.5 IU/kg+exenatide 0.1 μg); and 6) subcutaneously administered 1 IU/kg insulin aspart+1 IU/kg insulin glargine+1 μg/mouse exenatide in saline (Group 6: SC insulin aspart 1 IU/kg+insulin glargine 1 IU/kg+1 ug exenatide in saline).

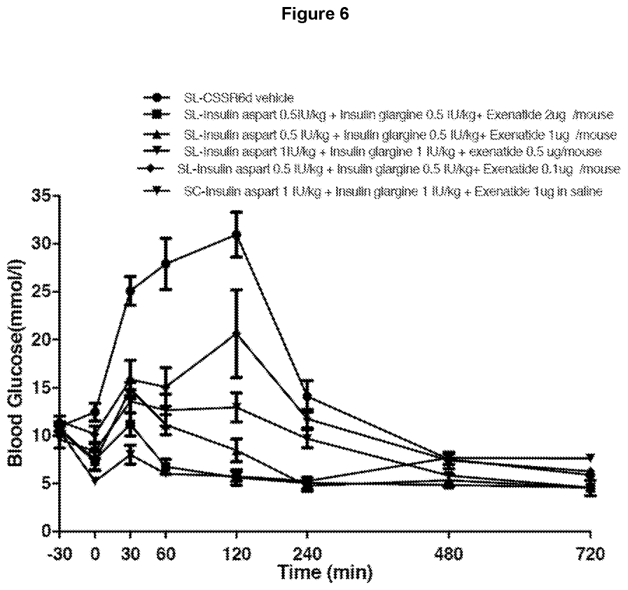

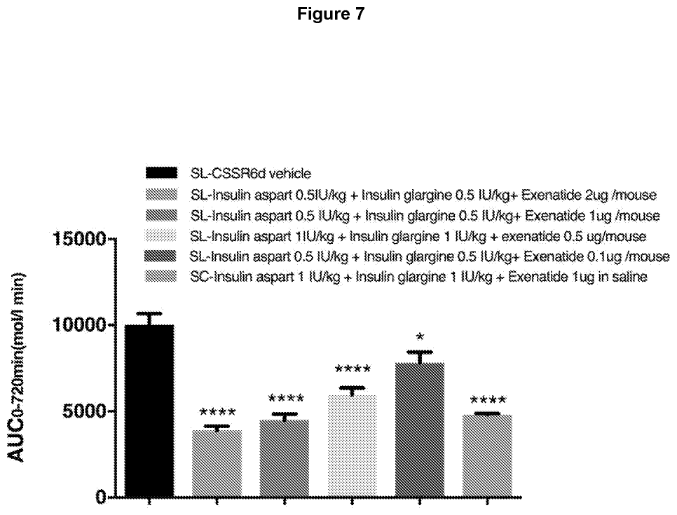

Blood glucose levels in each treatment group at −30 min, 0 min, 30 min, 60 min, 120 min, 240 min, 480 min and 720 min relative to the time of administration are shown in FIG. 6. Area under the curve analysis of blood glucose levels between 0-720 min (AUC₀₋₇₂₀ₘᵢₙ) for each treatment group are shown in FIG. 7.

The results of these experiments demonstrate that sublingual delivery systems of insulin aspart, insulin glargine and exenatide in oil formulations according to the present disclosure are comparable to the same dose subcutaneous delivery of insulin aspart and insulin glargine with exenatide. The results also show that sublingual administration of insulin in saline is not efficacious whereas the oil based formulations according to the invention are effective for the sublingual delivery systems of insulin aspart, insulin glargine and exenatide.

### Example 5—Sublingual Exenatide

Exenatide was dissolved in small quantity of water then mixed in an oil comprising 20% Vitamin E oil, 20% polyethylene glycol 200, and 60% flavouring oils with trace water base (“CSSR3 TPPM”)

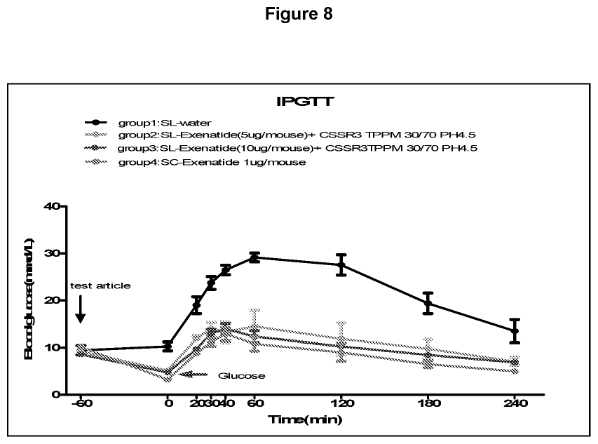

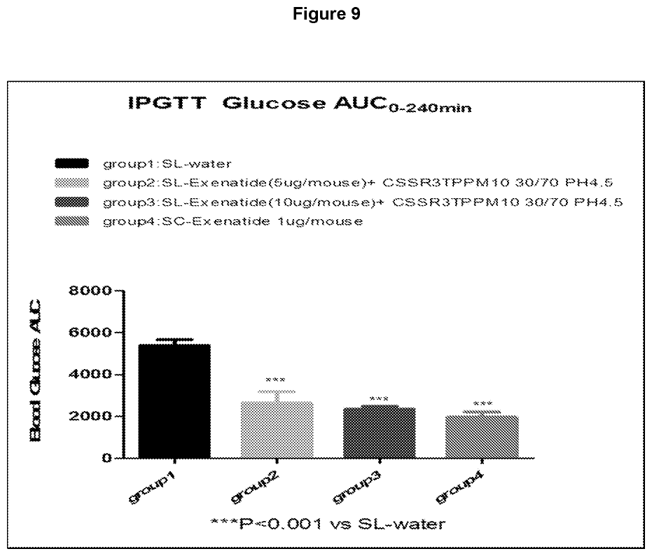

Male diabetic mice (strain C57/BL6) were sourced from Shanghai SLAC Laboratory Animal Co. Ltd. (Shanghai, China). For the experiments described below, mice of approximately 50 g were used. 5 μL of the resulting oil mixture comprising the active agent(s) was placed underneath the tongue of anesthetised diabetic mice. Blood glucose was measured at −30 min, 0 min, 30 min, 60 min, 120 min, 240 min, The mice were allowed access to food after 4 h. AUC analyses of blood glucose levels between 0-240 min (AUC₀₋₂₄₀ₘᵢₙ) for each treatment group are shown in FIGS. 8 and 9.

Three groups of five mice were treated. The treatment groups were administered as follows: 1) sublingual oil formulation placebo (Group 1: SL-CSS3TPPM); 2) subcutaneous administered 1 μg exenatide in dd water (Group 2: SC 1 μg in saline); 3) sublingually administered 5 μg exenatide in oil formulation (Group 3: SL exenatide 5 μg). 4) sublingually administered 10 μg exenatide in oil formulation (Group 4: SL exenatide 10 μg).

The results of these experiments (FIGS. 8 and 9) demonstrate that sublingual delivery systems of 5 or 10 ug exenatide in oil formulations according to the present disclosure are comparable to subcutaneous delivery of exenatide.

### Example 6—Sublingual Liraglutide and Sublingual Insulin Aspart/Insulin Glargine

Liraglutide was dissolved small quantity of water then mixed in an oil comprising 20% Vitamin E oil, 20% polyethylene glycol 200, and 60% flavouring oils (“CSSR6d”), and the mixture spun until homogenous. Insulin aspart, liraglutide and/or insulin glargine was dissolved in small quantity of dd water and added to an oil comprising 20% Vitamin E oil, 20% polyethylene glycol 200, and 60% flavouring oils (“CSSR6d”), and the mixture spun until homogenous.

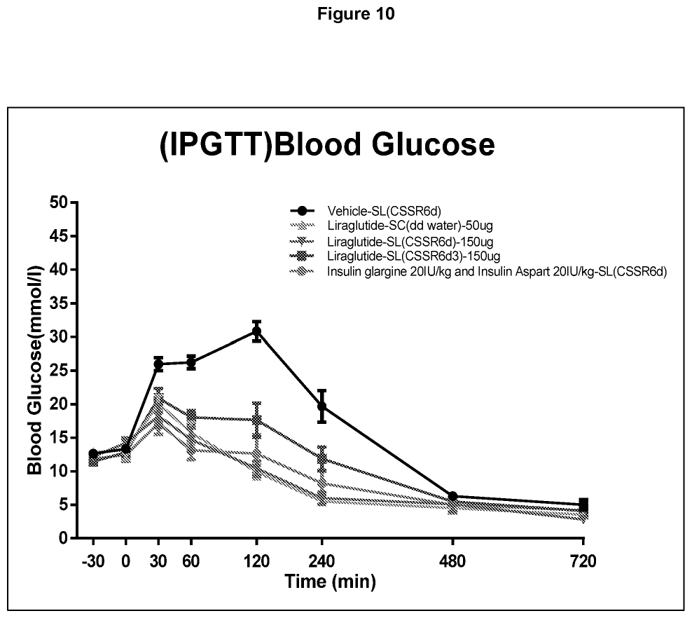

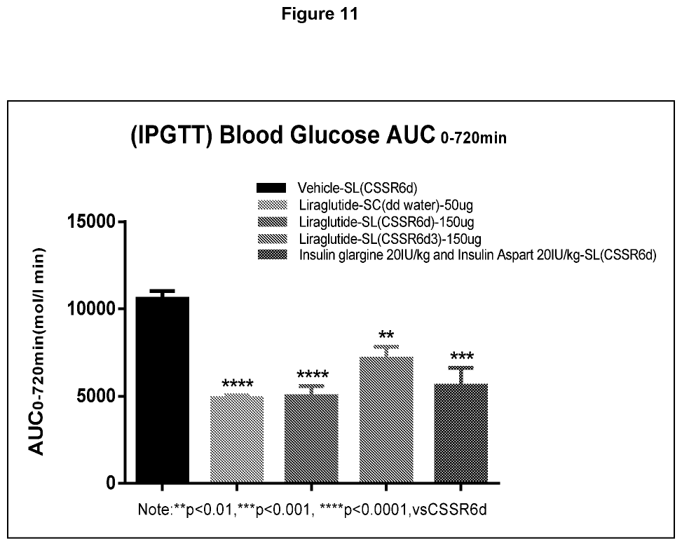

Male diabetic mice (strain C57/BL6) were sourced from Shanghai SLAC Laboratory Animal Co. Ltd. (Shanghai, China). For the experiments described below, mice of approximately 50 g were used. 5 μL of the resulting oil mixture comprising the active agent(s) was placed underneath the tongue of anesthetised diabetic mice. Blood glucose was measured at −30 min, 0 min, 30 min, 60 min, 120 min, 240 min, 480 min and 720 min. The mice were allowed access to food after 4 h. Area under the curve analysis of blood glucose levels between 0-720 min (AUC₀₋₇₂₀ₘᵢₙ) for each treatment group are shown in FIGS. 10 and 11.

Four groups of five mice were treated. The treatment groups were administered as follows: 1) sublingual oil formulation placebo (Group 1: SL-CSSR6d); 2) subcutaneous administered liraglutide in dd water (Group 3: SC liraglutide 50 μg 3) sublingually administered liraglutide in oil formulation (Group 2: SL liraglutide 150 μg); 4) sublingual administered insulin glargine and insulin in oil formulation CSSRd (Group 4: sublingual administered insulin glargine 20 IU/kg and insulin aspart 20 IU/kg in oil formulation CSSRd).

The results of these experiments (FIGS. 10 and 11) demonstrate that sublingual delivery systems of liraglutide in oil formulations and insulin glargine+aspart in oil formulation according to the present disclosure are comparable to subcutaneous delivery liraglutide.

### Example 7—Sublingual Resveratrol

Reservatrol was dissolved in an oil comprising 20% Vitamin E oil, 20% polyethylene glycol 200, and 60% flavouring oils (“CSSR6d”) and the mixture spun until homogenous for sublingual administration. Resveratrol was suspended in 50% sugar solution and the mixture spun until homogenous for oral ingestion.

Two groups of each three SD rats were treated. The treatment groups were administered as follows: 1) sublingual resveratrol in oil formulation (Group 1: SL-resveratrol 10 mg in oil formulation); 2) oral administered resveratrol in sugar syrup (Group 2: OP resveratrol 10 mg in sugar syrup). Nine blood sampling time points up to 24 hours post dosing per anima (0, 0.125, 0.25, 0.5, 1, 2, 4, 8, 12 hours). Plasma samples bio analysis was performed with LC-MS/MS method.

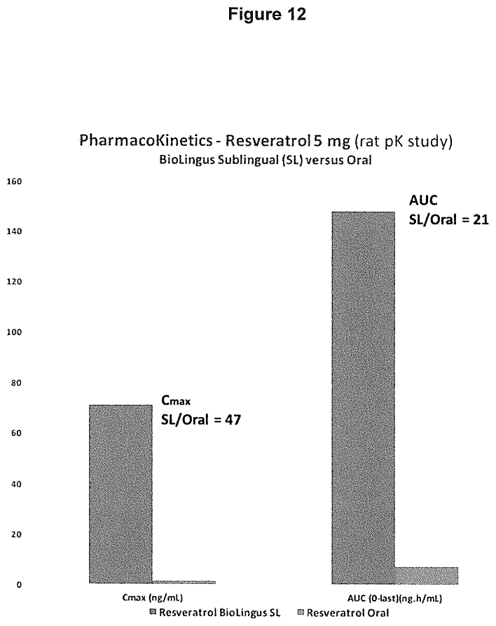

The results of this experiment (FIG. 12) demonstrate that sublingual delivery of resveratrol in oil formulations according to the present disclosure are 20 to 40 times more absorbable than orally administered resveratrol.

1. An delivery system formulated for sublingual and/or buccal delivery comprising:
at least one functional oil;
at least one surfactant; and
at least one pharmaceutically active agent.

2. The delivery system of claim 1, wherein the pharmaceutically active agent is a water insoluble or sparingly soluble macromolecule, peptide or protein.

3. The delivery system of claim 1 or claim 2, wherein the pharmaceutically active agent is a water-in-oil soluble macromolecule, peptide or protein.

4. The delivery system of any one of the preceding claims, wherein the delivery system is delivered sublingually.

5. The delivery system of any one of claims 1 to 3, wherein the delivery system is delivered buccally.

6. The delivery system of any one of the preceding claims, wherein the functional oil comprises vitamin oil, palm oil, rice bran oil, marine oil, citrus oil, flax oil, castor oil, sunflower oil, olive oil, soybean oil, corn oil, walnut oil, peanut oil, almond oil, or an oil derived from rice, wheat, barley, rye, oats, saw palmetto or annatto.

7. The delivery system of claim 6, wherein the functional oil comprises vitamin oil.

8. The delivery system of claim 7, wherein the functional oil comprises vitamin E oil.

9. The delivery system of any one of the preceding claims, wherein the functional oil comprises one or more tocotrienols.

10. The delivery system of any one of the preceding claims, wherein the functional oil comprises one or more tocopherols.

11. The delivery system of any one of the preceding claims, wherein the surfactant is selected from the group consisting of a monoglyceride, lecithin, polyethylene glycol, propylene glycol, a glycerol derivative, a sugar alcohol, polyethoxylated castor oil, polyoxyethylene sorbitan monolaurate, polyoxyethylene sorbitan monoleate, tocopheryl polyethylene glycol succinate, a polyethoxylated sorbitan ester, a sorbitan ester, a triglyceride, a gelatin, a protein, a gel formulation, and mixtures thereof.

12. The delivery system of claim 11, wherein the surfactant is polyethylene glycol.

13. The delivery system of claim 12, wherein the surfactant is polyethylene glycol 200.

14. The delivery system of any one of the preceding claims, wherein the pharmaceutically active agent is selected from the group consisting of a hormone, a hormone analogue, a hormone mimetic, a glucagon-like peptide-1 agonist, a cytokine, an interleukin, interferon, a vaccine, an antibody, an allergen, a scaffold protein, and an oil soluble plant extract, and combinations thereof.

15. The delivery system of claim 14, wherein the pharmaceutically active agent is a peptide hormone, a peptide hormone analogue or a peptide hormone mimetic.

16. The delivery system of claim 15, wherein the at least one active agent is a glucagon-like peptide-1 agonist.

17. The delivery system of claim 16, wherein the glucagon-like peptide-1 agonist is exenatide or liraglutide.

18. The delivery system of claim 15, wherein the pharmaceutically active agent is insulin or an analogue or mimetic thereof.

19. The delivery system of claim 18, wherein the insulin is insulin aspart and/or insulin glargine.

20. The delivery system of claim 14, wherein the pharmaceutically active agent is a mixture of exenatide and insulin.

21. The delivery system of any one of the preceding claims, further comprising at least one additional oil or oil mixture.

22. The delivery system of claim 21, wherein the additional oil is an essential oil.

23. The delivery system of claim 22, wherein the essential oil is selected from the group consisting of camphor oil, menthol, eucalyptus oil, orange oil, mandarin oil, peppermint oil, clove oil, coconut oil, chlorbutol, arachis oil, lemon oil, spearmint oil, citronella oil, geranium oil, thymol, and combinations thereof.

24. The delivery system of any one of the preceding claims, wherein the delivery system is in the form of a spray, drops, or is absorbed onto a solid carrier.

25. A composition formulated for the delivery of pharmaceutically active agents via the sublingual and/or buccal mucosa, wherein the composition comprises the delivery system of any one of the preceding claims.

26. A method for treating diabetes, for regulating blood glucose levels and/or for preventing or treating hyperglycaemia in a subject, the method comprising sublingually and/or buccally administering to the subject an effective amount of the delivery system of any one of claims 1 to 24.

27. A method for treating diabetes, for regulating blood glucose levels and/or for preventing or treating hyperglycaemia in a subject, the method comprising sublingually and/or buccally administering to the subject an effective amount of the composition of claim 25.

28. The method of claim 26 or 27, wherein the active agent is insulin and/or an analogue or mimetic thereof.

29. The method of claim 28, wherein the insulin analogue comprises insulin aspart and/or insulin glargine.

30. The method of claim 26 or 27, wherein the active agent is a glucagon-like peptide-1 agonist.

31. The method of claim 30, wherein the glucagon-like peptide-1 agonist is exenatide or liraglutide.

32. The method of claim 26 or 27, wherein the active agent is a mixture of a glucagon-like peptide-1 agonist and insulin, or an analogue or mimetic thereof.

33. The method of claim 32, wherein the insulin analogue comprises insulin aspart and/or insulin glargine, and the glucagon-like peptide-1 agonist is exenatide or liraglutide.
